(19) United States
(12) Patent Application Publication
(10) Pub. No.: US 2007/0135829 A1
(43) Pub. Date: Jun. 14, 2007

(54) Intra-gastric balloon coated in parylene, a method of fabricating such a balloon and of using parylene to coat an intra-gastric balloon

(76) Inventor: Pascal Paganon, Villeurbanne (FR)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/550,783

(22) PCT Filed: Mar. 31, 2004
(86) PCT No.: PCT/FR04/00810

§ 371 Date: Nov. 9, 2006

(30) Foreign Application Priority Data
Mar. 31, 2003 (FR) 03/03948

(51) Int. Cl. A61M 29/00

(52) U.S. Cl. 606/192

(57) ABSTRACT

An expandable intra-gastric balloon (1) for treating obesity. The balloon can be implanted in the stomach of a patient in order to reduce the volume of the stomach. The balloon (1) comprises at least one flexible bag (2) presenting an inside face (2A) and an opposite, outside face (2B). The inside and outside faces (2A, 2B) form the surface of the at least one flexible bag (2). At least a portion of the surface (2A, 2B) is covered by a coating (6, 6′) comprising parylene.

### TECHNICAL FIELD

The present invention relates to the technical field of artificial devices for treating obesity, in particular morbid obesity, and more particularly devices that consist in artificially reducing the volume of the gastric cavity in order to give the patient a sensation of being sated rapidly.

The present invention relates to an expandable intra-gastric balloon for treating obesity, which balloon is designed to be implanted in the stomach of a patient in order to reduce the volume of the stomach, said balloon comprising at least one flexible bag presenting an inside face and an opposite, outside face, said inside and outside faces forming the surface of the at least one flexible bag.

The invention also relates to a method of fabricating an expandable intra-gastric balloon for treating obesity, said balloon being for implanting in the stomach of a patient in order to reduce the volume of the stomach, in which at least one flexible bag is provided or made that presents an inside face and an opposite outside face, said inside and outside faces forming the surface of the at least one flexible bag.

The invention also relates to a novel use of parylene.

### PRIOR ART

In order to treat patients suffering from obesity, in particular those presenting a weight/size ratio that does not require the use of invasive surgical methods and devices that are expensive and traumatizing, such as surgically implanting a gastric ring, and also for treating patients whose excess weight is considered to constitute a risk in terms of a surgical intervention, it is known to implant a foreign body directly in the stomach of the patient, said body being of a volume that is sufficient to reduce the space available for food, while also reducing the rate at which food passes through.

Such foreign bodies are implanted, and are generally in the form of so-called “intra-gastric” balloons, formed by a flexible bag made of biocompatible elastomer material and implanted directly in the patient's stomach.

The balloon has an orifice with a valve installed therein, these two elements forming connection means in which the surgeon installs a connection member prior to implanting the balloon in its non-expanded state, which member is generally a catheter connected to a source of fluid (physiological liquid and/or gas), making it possible, subsequently, to inflate or expand the balloon inside the stomach.

Such intra-gastric balloons are well known and they provide results that are of interest in terms of losing weight, since they reduce the rate at which food passes through and they contribute effectively to quickly generating a sensation of being sated, but nevertheless they suffer from drawbacks that are not negligible.

In particular, putting them in place, and in particular handling them and expanding them, can sometimes be difficult.

The balloon is generally positioned in the stomach as follows:

the balloon in its non-expanded configuration is folded (or rolled or twisted) in such a manner as to present a shape that is generally oblong;

the balloon folded in this way is placed inside a cover for holding it in its folded configuration; said cover is made of an elastomer material and is provided with regions of weakness, such as slots;

the assembly formed by the cover containing the folded bag is inserted into the patient's stomach;

the balloon is inflated using the above-mentioned catheter, thereby expanding the cover of elastomer material until it bursts, with this being made easier by its slots of weakness; and

the catheter and the cover are then withdrawn from the patient's body, leaving the balloon on its own in the stomach.

As a general rule, the balloon and the cover are both made of silicone, a material that presents excellent properties of elasticity, strength, and biocompatibility. However, silicone also presents contact that is sticky, i.e. it is somewhat tacky to touch, thus making it difficult to handle the balloon, particularly during the step of folding it. This difficulty of handling during folding prevents the volume occupied by the balloon in the folded state being optimized, in particular prevents optimization of the cross-section of this oblong volume, even though it is specifically desired to minimize this cross-section so as to facilitate inserting the balloon via the natural passageway into the patient's stomach, thus providing the patient with better comfort and better safety.

In addition, this tacky contact of silicone does not encourage inflation of the balloon and release of the balloon from its cover. The stickiness between the cover and the balloon slows down and impedes proper operation of the stages of inflating and releasing the balloon. This drawback is made worse by the sticky nature of silicone encouraging the balloon to stick to itself when it is in its folded configuration.

To mitigate this effect of silicone being sticky, it is known to use talc or sodium bicarbonate. However those substances present the disadvantage of polluting the white room in which the balloon is fabricated, and therefore require fabrication procedures to be put into place that are complex and expensive.

Furthermore, prior art intra-gastric balloons are difficult to subject to gamma ray sterilization treatment without running the risk of being damaged thereby due to the bag of the balloon folding over onto itself.

Another problem with prior art intra-gastric balloons is that they generally present a certain amount of porosity which will allow the fluid contained in the balloon (gas or liquid) to leak out gradually, thereby progressively diminishing the volume of the balloon, and thus reducing its therapeutic effective.

This problem is particularly inconvenient when the intra-gastric balloon is filled with gas only. However in order to minimize the weight of the balloon it is most desirable for it to be filled with gas and not with liquid.

### SUMMARY OF THE INVENTION

Consequently, the objects given to the invention seek to remedy the various drawbacks listed above and to propose a novel expandable intra-gastric balloon for treating obesity in which fabrication and implantation, in particular folding and expansion, are particularly simplified and fast.

Another object of the invention is to provide a novel intra-gastric balloon presenting a longer duration of therapeutic effectiveness.

Another object of the invention is to provide a novel intra-gastric balloon that is particularly strong with reduced losses of fluid (and in particular of gas).

Another object of the invention is to propose a novel intra-gastric balloon of simplified design that presents good resistance in general, in particular good mechanical strength.

Another object of the invention is to propose a novel intra-gastric balloon that presents excellent dimensional regularity.

Another object of the invention seeks to propose a novel intra-gastric balloon which, although of sufficient volume, is nevertheless particularly light in weight and well accepted by the patient.

Another object of the invention seeks to propose a novel method of fabricating an intra-gastric balloon that is particularly simple and fast to implement, while making it possible to obtain a balloon that presents thickness of excellent uniformity.

Another object of the invention seeks to propose a novel use for parylene.

The objects given to the invention are achieved with the help of an expandable intra-gastric balloon for treating obesity, for implanting in the stomach of a patient in order to reduce the volume of the stomach, said balloon comprising at least one flexible bag presenting an inside face and an opposite outside face, said inside and outside faces forming the surface of the at least one flexible bag, the balloon being characterized in that at least a portion of said surface is covered by a coating comprising parylene.

The objects given to the invention are also achieved with the help of a method of fabricating an expandable intra-gastric balloon for treating obesity, said balloon being designed to be implanted in the stomach of a patient in order to reduce the volume of the stomach, in which method at least one flexible bag is provided or made that presents an inside face and an opposite, outside face, said inside and outside faces forming the surface of the at least one flexible bag, the method being characterized in that it comprises a deposition step in which at least a portion of said surface is covered in a coating comprising parylene.

Finally, the objects given to the invention are achieved with the help of the use of parylene as a coating for an intra-gastric balloon.

### BRIEF DESCRIPTION OF THE DRAWINGS

Other objects and advantages of the invention appear better on reading the following description and with the help of the accompanying drawings given purely by way of non-limiting illustration, and in which:

FIG. 1 is a perspective view showing an intra-gastric balloon in accordance with the invention in its maximally expanded position, and fitted with a tubular connection member;

FIG. 2 is a diagrammatic cross-section view showing a step of the method of fabricating an intra-gastric balloon having two bags, in accordance with the invention; and

FIG. 3 is a diagrammatic cross-section view showing a flexible bag for use in fabricating an intra-gastric balloon of the invention having a single bag.

### BEST METHOD OF PERFORMING THE INVENTION

FIG. 1 shows an intra-gastric balloon 1 in accordance with the invention. Such a balloon is designed to treat obesity, and for this purpose it is implanted in the stomach of a patient in order to reduce the volume of the stomach, insofar as the balloon occupies a major fraction of the space that would otherwise be available for food.

The intra-gastric balloon 1 in accordance with the invention is expandable, i.e. it is arranged to occupy both a folded or relaxed configuration (not shown in the figures) in which it occupies a small volume, making it easier to implant, and secondly an expanded configuration of substantially predetermined volume, e.g. of the order of 600 millimeters (mL), corresponding to its volume in use, as shown in particular in FIG. 1.

Preferably, the expandable nature of the intra-gastric balloon 1 is obtained firstly by making it out of flexible materials, e.g. elastomers, and secondly by having recourse to one or more inflation fluids that are introduced into the balloon in order to bring it into its expanded configuration.

Nevertheless, without going beyond the ambit of the invention, it is entirely possible to envisage the intra-gastric balloon 1 in accordance with the invention being formed by a structure that does not present a flexible nature, but rather is rigid or semirigid in nature. In this context, it is possible to envisage the balloon 1 being constituted by a deployable structure that is expanded not by delivering fluid, but by an elastic effect or by implementing materials having shape memory.

In general, the intra-gastric balloon 1 in accordance with the invention is implanted in a manner that is conventional and well-known to the person skilled in the art by passing from the mouth and through the esophagus while in its folded or relaxed state. The expansion and positioning and release of the balloon take place at the end of the surgical operation once the intra-gastric balloon 1 is properly positioned in the patient's stomach.

The intra-gastric balloon 1 in accordance with the invention is thus preferably a balloon that is arranged to be implanted in the patient's stomach solely by the natural passageways. In other words, the balloon 1 is preferably designed to be put into place solely by endoscopy.

The intra-gastric balloon 1 in according with the invention comprises at least one flexible bag.

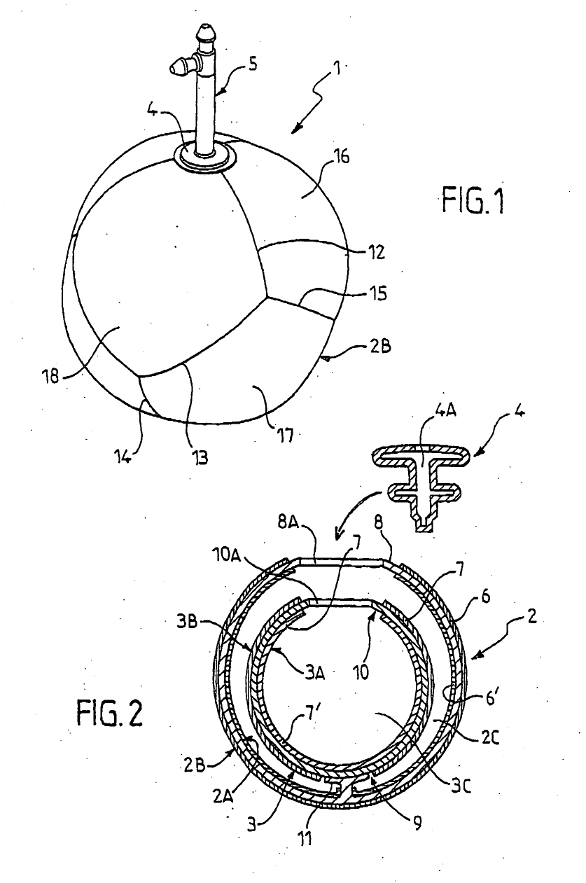

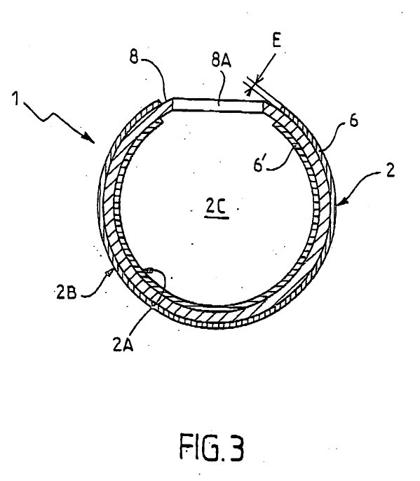

The description below makes reference simultaneously firstly to a first embodiment of a balloon 1 in accordance with the invention in which the balloon comprises a single flexible bag 2 (FIG. 3), and secondly to a second embodiment in which the balloon comprises two flexible bags 2, 3 (FIG. 2). In the context of the invention, it is possible to provide for some greater number of bags (e.g. three, four, or even more), each bag being capable of being inflated with a different fluid. For a balloon having two bags as shown in FIG. 2, it is thus possible to inflate one of the bags with physiological liquid, while inflating the other bag with a gas that is of lower density, e.g. air. This makes it possible for a given total volume of the intra-gastric balloon 1 to obtain a weight that is less than that of conventional single-bag balloons.

This disposition having two or more bags thus makes it possible to reduce the total weight of the intra-gastric balloon once implanted in the patient's stomach, thereby making it more acceptable to the organism and reducing side effects.

In the embodiment shown in FIG. 2, the balloon of the invention comprises first and second flexible bags 2 and 3, said second bag 3 being placed inside the first bag 2, so that the first bag is thus of greater outside volume, at least in the expanded state.

Preferably, the second bag 3 forms an internal bag of general shape that may be identical or different from that of the bag 2 that forms the main bag.

In this embodiment, it is preferable for the second bag 3 to be filled with gas, e.g. air, while the first bag 2 is filled with liquid, e.g. physiological water.

Advantageously, the second bag 3 is placed substantially concentrically inside the first bag 2, and is thus surrounded over substantially its entire outside surface by the liquid of the bag 2. This provides good sealing for the second bag 3, thereby reducing any risk of the gas contained therein leaking out.

It is also possible to envisage the second bag 3 being inflated directly and thus acting as an “inner tube” for the first bag 2, which then expands solely under the effect of the outward force exerted by the second bag 3.

It is also possible to envisage the first and second bags 2 and 3 being adjacent and interconnected via a common face, so that in combination, e.g. by being stuck together, the two bags form one balloon.

In the variant shown in FIG. 2, spacing is maintained between the first and second bags 2 and 3 with the help of holding means 4, 11 serving to hold the two bags 2 and 3 apart from each other. The holding means 4, 11 preferably comprise spacers that hold and secure the two bags 2 and 3 at a distance apart from each other.

Said at least one flexible bag 2, 3 presents an inside face 2A, 3A and an opposite, outside face 2B, 3B. Said at least one bag 2, 3 defines a predetermined inside volume 2C, 3C, the inside face 2A, 3A being situated facing said inside volume 2C, 3C, while the outside face 2B, 3B faces outwards from said inside volume 2C, 3C.

Said inside and outside faces 2A, 3A and 2B, 3B form the surface of the at least one flexible bag 2, 3.

Advantageously, said at least one flexible bag 2, 3 is made on the basis of an elastomer material.

Preferably, the at least one flexible bag 2, 3 is made of silicone.

In conventional manner, said at least one flexible bag 2, 3 is also provided with connection means 4 including at least one orifice and valve system for receiving a connection member 5 for connection at least one source of the corresponding fluid (not shown in the figures) in order to expand said at least one bag 2, 3 in the stomach by filling it with fluid.

For a balloon having two bags as shown in FIG. 2, each bag 2, 3 may advantageously be provided with distinct connection means, so that each bag can be connected to a distinct fluid source, namely a first fluid source and a second fluid source.

This makes it possible, for example, to fill the bag 3 with gas and the bag 2 with liquid.

According to an important characteristic of the invention, and as shown more particularly in FIGS. 2 and 3, at least a fraction of the surface of said at least one flexible bag 2, 3 is covered in a coating 6, 6′, 7, 7′ that comprises parylene.

In other words, with reference to the single-bag balloon shown in FIG. 3, the balloon is in accordance with the invention providing at least a fraction of its inside face 2A is covered by the coating 6′ comprising parylene, or providing at least a fraction of its outside face 2B is covered by the coating 6 comprising parylene, or indeed providing a fraction at least of both its inside face 2A and its outside face 2B is covered by the coating 6, 6′ comprising parylene.

For a balloon having more than one bag, and in particular for a balloon as shown in FIG. 2 comprising first and second bags 2 and 3, in the context of the invention it is possible for at least a portion of the surface of only the first bag 2 to be covered in the coating 6, 6′ comprising parylene, or for at least a portion of the surface of the second bag 3 only to be covered by the coating 7, 7′ comprising parylene, or indeed for at least a portion of the surface of the first bag 2 and at least a portion of the surface of the second bag 3 to be covered by the coating 6, 6′, 7, 7′.

Advantageously, the balloon 1 comprises first and second flexible bags 2 and 3, said second bag 3 being placed inside the first bag 2, the first bag 2 having its inside and outside faces 2A and 2B partially or completely covered in a coating essentially based on parylene. Preferably, the second bag 3 also has its inside and outside faces 3A and 3B partially or completely covered in a coating essentially based on parylene.

It is also possible to envisage the first and second bags 2 and 3 being made out of different materials. For example the first bag may be made of silicone while the second bag is made of a material that is gasproof.

Parylene is the generic name of a series of polymers developed by Union Carbide Corporation. There are three major families in this series of polymers known respectively by the following names: parylene N, parylene C, and parylene D.

Parylene N, which is the basic polymer of the series, is poly-para-xylylene, which is a highly crystalline linear substance.

Parylenes C and D are derived from parylene N.

Advantageously, the coating 6, 6′, 7, 7′ is constituted by parylene, and more preferably by parylene C.

Preferably, the parylene used is that sold under the commercial name Galxyl (registered trademark) by the supplier Comelec.

Preferably, the surface of at least one flexible bag 2, 3 is entirely covered by the coating 6, 6′, 7, 7′ with the exception of the zones 8, 9, and 10 acting as interfaces with the devices 4, 11 fitted to the bag 2, 3.

By way of example, these devices may comprise connection means 4. Such a connection means is fitted to the flexible bag(s) 2, 3, e.g. by adhesive, at a peripheral edge 8, 10 of an orifice 8A, 10A formed through the corresponding bags 2, 3.

Other devices for fitting to the bags 2, 3 include, for a two-bag balloon, the spacer 11, e.g. made integrally with the first bag 2, and stuck to a portion 9 of the outside face 3B of the second bag 3.

Advantageously, the thickness E of the coating 6, 6′, 7, 7′ lies in the range 0.2 micrometers (μm) to 100 μm.

More preferably, the thickness E of the coating 6, 6′, 7, 7′ lies in the range 0.5 μto 6 μm.

Still more preferably, the thickness E lies in the range 1.5 μm to 6 μm.

In the context of the invention, it is entirely possible for the thickness E to vary over the surface, and/or for the thickness E to vary between the inside and outside surfaces 2A & 2B or 3A, 3B of a given bag, or for the thickness to vary between two distinct bags 2, 3.

Nevertheless, it is preferable for the coating 6, 6′, 7, 7′ to be uniform, i.e. for its thickness E to be constant.

Advantageously, and as shown in FIG. 1, the outside face 2B of the balloon of the invention is shaped so as to co-operate with the wall of the stomach (not shown) with which the balloon comes into contact once in position inside the body of the patient, to define passage channels 12, 13, 14, and 15 between the zone situated on the upstream side of the balloon 1 relative to the food transit direction towards the zone of the stomach situated downstream from said balloon. The channels 12, 13, 14, and 15 form an array that branches at more than two points so as to constitute a tree structure for food passing from the upstream zone of the stomach towards the downstream zone.

The term “an array that branches at more than two points” is used herein to mean that junctions or subdivisions are provided in the channels at at least three points in the array of channels.

More particularly, the outside face 2B corresponds to a plurality of bulges 16, 17, 18 arranged relative to one another in such a manner that the channels 12, 13, 14, 15 are defined firstly by the interstices between the bulges 16, 17, and 18, and secondly by the wall of the stomach that comes into contact with the tops of said bulges 16, 17, and 18.

Preferably, the bulges 16, 17, and 18 are arranged relative to one another in the configuration of a truncated icosahedron (not shown in the figures).

Such an arrangement makes it possible to lengthen the total time taken to digest food, and thus to prolong the effect of feeling sated, while significantly overcoming any “check valve” effect between the balloon and the stomach wall.

The invention also provides a method of fabricating an expandable intra-gastric balloon for treating obesity, said balloon being for implanting in the stomach of a patient in order to reduce the volume of the stomach.

In accordance with the method of the invention, a first step is implemented in which at least one flexible bag 2, 3 is provided or made, e.g. with the help of an injection-molding method or a dip coating method, the bag presenting an inside face 2A, 3A and an opposite, outside face 2B, 3B, said inside and outside faces 2A, 3A and 2B, 3B forming the surface of the at least one flexible bag 2, 3.

According to an important characteristic of the invention, said method includes a subsequent step of depositing a coating 6, 6′, 7, 7′ comprising parylene, in which at least a portion of said surface of the flexible bag 2, 3 is covered.

Advantageously, said coating is constituted by parylene C.

Advantageously, the coating 6, 7 is put into place on the at least one bag by rarefied gas deposition. Such deposition could be referred to as vapor deposition polymerization (VDP).

In simplified manner, the method of depositing parylene that is implemented in the context of the invention comprises the following steps:

the solid dimer of di-para-xylylene is vaporized;

the gaseous dimer obtained from the preceding step is subjected to pyrolysis, thereby converting it into a reactive monomer, para-xylylene; and

the gaseous monomer obtained by the preceding step is then introduced into a deposition chamber where it is adsorbed onto the surface that is to be covered and polymerizes on said surface.

This method thus makes it possible to cover the entire exposed surface of the flexible bag 2, 3 in question in uniform manner, naturally with the exception of the zones 8, 9, and 10 that are not to be covered, which zones are therefore protected by protection means, such as a protective film that is removed after the operation of depositing parylene.

Finally, the invention relates to using parylene as a coating for an expandable intra-gastric balloon for treating obesity, said balloon being for implanting in the stomach of a patient in order to reduce the volume of the stomach.

The invention also relates as such to a method of sterilizing medical equipment, in particular an intra-gastric balloon, making use of the protective and/or barrier nature of polymer coatings that make it possible, surprisingly, to subject the intra-gastric balloon to the sterilizing effect of a flux of gamma radiation without adversely influencing the future properties of the balloon material.

Thus, in general, the method of sterilizing an intra-gastric balloon in accordance with the invention is characterized in that prior to subjecting the intra-gastric balloon to gamma radiation, the balloon is covered in a protective coating based on polymer.

The step of applying the polymer coating can take place at any moment in the balloon-fabrication line, but preferably takes place at the end of the line, or in any event at a step that enables the protective properties of the polymer to be conserved as well as possible during the subsequent step.

Coating may be applied on the inside and/or outside faces of the balloon, or on the outside face only, providing a good protective effect is obtained.

Advantageously, the protective polymer is based on parylene, i.e. it contains a concentration of parylene that is sufficient and necessary for obtaining the desired protective effect.

In its preferred application, the invention also provides a method of fabricating an intra-gastric balloon in accordance with the invention, in which, after the deposition step consisting in covering at least a fraction of the surface 2A, 3A, 2B, 3B of the balloon in a coating comprising parylene, the balloon is subjected to a sterilization stage comprising a step of subjecting it to gamma radiation.

The novel uses of parylene as a protective coating for an intra-gastric balloon are at the origin not only of novel properties of leaktightness or of preventing silicone from sticking, but also novel properties of providing protection against gamma radiation.

Thus, implementing the invention makes it possible to obtain an intra-gastric balloon that is substantially leaktight, that turns out to be particularly easy to fold up very tight, and that easily releases its protective cover on being expanded in the stomach.

The good anti-stick properties obtained for the balloon make it possible to avoid having recourse to additional substances such a talc or sodium bicarbonate, thereby greatly simplifying fabrication while also greatly reducing environmental harm.

The balloons that are obtained can also be subjected to a sterilization stage comprising a step of being subjected to gamma radiation, without running the risk of degrading the constituent(s) of the balloon, because of the barrier and/or protective effect of the coating made of polymer, and specifically in the present example of parylene.

These properties are particularly advantageous for a balloon having a plurality of bags, and/or for a balloon having a bumpy surface.

### SUSCEPTIBILITY OF INDUSTRIAL APPLICATION

Industrial application of the invention lies in making and using intra-gastric balloons for treating obesity.

1.-16. (canceled)

17. An expandable intra-gastric balloon for treating obesity, for implanting in the stomach of a patient in order to reduce the volume of the stomach, said balloon comprising:
at least one flexible bag presenting an inside face and an opposite, outside face, said inside and outside faces forming the surface of the at least one flexible bag, wherein at least a portion of said surface is covered by a coating comprising parylene.

18. The balloon of claim 17, wherein said at least one flexible bag is made from silicone.

19. The balloon of claim 17, wherein the coating is constituted by parylene C.

20. The balloon of claim 17, wherein the surface of the at least one flexible bag is covered entirely by the coating, with the exception of zones acting as interfaces with devices fitted to the at least one flexible bag.

21. The balloon of claim 17, wherein the thickness of the coating lies in the range of 0.2 μm to 100 μm.

22. The balloon of claim 21, wherein the thickness of the coating lies in the range of 1 μm to 50 μm.

23. The balloon of claim 17, wherein said at least one flexible bag is configured to connect to a corresponding fluid source in order to expand said at least one bag in the stomach by being filled with fluid.

24. The balloon of claim 17, wherein said at least one flexible bag comprises a first flexible bag and a second flexible bag, said second flexible bag being disposed inside the first flexible bag.

25. The balloon of claim 24, wherein said second flexible bag is configured to be connected to a second fluid source in order to expand said second bag in the stomach by being filled with fluid.

26. The balloon of claim 25, wherein the surface of each of said first and second flexible bags is covered, at least in part, by the coating comprising parylene.

27. A method of fabricating an expandable intra-gastric balloon for treating obesity, said balloon being designed to be implanted in the stomach of a patient in order to reduce the volume of the stomach, comprising:
depositing a coating of parylene on at least a portion of a surface of at least one inflatable bag of said balloon, the at least one flexible bag having an inside face and an opposite, outside face, said inside and outside faces forming the surface.

28. The method of claim 27, wherein the step of depositing the coating of parylene further comprises:
depositing the coating of parylene on at least one bag by rarefied gas deposition.

29. The method of claim 27, further comprising:
fabricating the flexible bag from an elastomer material.

30. The method of claim 27, further comprising the step of:
sterilizing the balloon by subjecting the balloon to gamma radiation.

31. A method comprising:
coating an intra-gastric balloon with parylene.

32. A method of sterilizing an intra-gastric balloon, comprising:
prior to subjecting the balloon to gamma radiation, covering the balloon in a protective coating based on a polymer.

33. The method of claim 32, wherein the step of covering the balloon further comprises:
covering the balloon in the protective coating based on the polymer, the polymer being parylene.

34. The method of claim 33, further comprising the step of:
subjecting the balloon to gamma radiation.

35. The method of claim 33, further comprising:
applying the protective coating on an outside face of the balloon.

36. The method of claim 33, wherein the step of covering the balloon in the protective coating further comprises:
covering the balloon with the protective coating of parylene having a concentration of parylene sufficient for protecting the balloon against gamma radiation.
